(19) United States
(12) Patent Application Publication
(10) Pub. No.: US 2015/0119606 A1
(43) Pub. Date: Apr. 30, 2015

(54) Process for production of vanillin and vanillin derivatives

(71) Applicants: RHODIA OPERATIONS, Paris (FR); CENTRE NATIONAL DE LA RECHERCHE SCIENTIFIQUE (C.N.R.S.), Paris (FR)

(72) Inventors: Matthieu Corbet, Shanghai (CN); Pascal Metivier, Shanghai (CN); Floryan Decampo, Shanghai (CN)

(21) Appl. No.: 14/398,981

(22) PCT Filed: May 6, 2013
(86) PCT No.: PCT/CN2013/075203

§ 371 Date: Nov. 5, 2014

(30) Foreign Application Priority Data
May 7, 2012 (CN) PCT/CN2012/075131

(51) Int. Cl. C07C 45/54

(52) U.S. Cl. CPC C07C 45/54
USPC 568/433

(57) ABSTRACT

Disclosed is a process to produce vanillin or vanillin derivatives carrying out a one-step reaction starting from guaiacol or guaiacol derivatives and at least a superacid.

The present invention concerns a process to produce vanillin or vanillin derivatives carrying out a one step reaction starting from guaiacol or guaiacol derivatives and at least a superacid. The present invention relates to the organic chemistry.

### PRIOR ART

Vanillin is a phenolic aldehyde, an organic compound with the molecular formula C₈H₈O₃. Its functional groups include aldehyde, ether, and phenol. Synthetic and natural vanillin or its vanillin derivatives are used as a flavoring agent, notably in foods, beverages, and pharmaceuticals.

Vanillin was first synthesized from eugenol, found in clove oil, in 1875. Less than 20 years after it was first identified and isolated. Vanillin was commercially produced from eugenol until the 1920s. Later it was synthesized from lignin-containing “brown liquor”, a byproduct of the sulfite process for making wood pulp. Counter-intuitively, even though it uses waste materials, the lignin process is no longer popular because of environmental concerns, and today most vanillin is produced from the petrochemical raw material guaiacol. Several routes exist for synthesizing vanillin from guaiacol.

At present, the most significant of these is the two-step process practiced by Rhodia since the 1970s, in which guaiacol reacts with glyoxylic acid by electrophilic aromatic substitution. The resulting vanillylmandelic acid is then converted via 4-hydroxy-3-methoxyphenylglyoxylic acid to vanillin by oxidative decarboxylation.

Such an effective process is however not atom-economical, is carried out in 2 steps and has a non negligible carbon footprint. Thus, there is a need to develop a more environmentally friendly one step process in this technical field.

Although synthetic pathways to introduce a formyl group on aromatic compounds are well established, only a handful of them report on the direct formylation of phenols in decent yields, mainly due to problems of regioselectivity. The latter methods, however, all suffer from using expensive and/or very toxic, corrosive reagents in large quantities under harsh conditions (Saint-Jalmes, L.; Rochin, C.; Janin, R.; Morel, M. Industrial Chem. Library 1996, 8, 325-335. Schiraldi, D. A.; Kenvin, J. C. U.S. Ser. No. 00/591,0613A, 1999. Kantlehner, W. Eur. J. Org. Chem. 2003, 2530-2546 and “For a review on formylating agents”, Olah, G. A.; Ohannesian, L.; Arvanaghi, M. Chem. Rev. 1987, 87, 671-686 and Bagno, A.; Kantlehner, W.; Scherr, 0.; Vetter, J.; Ziegler, G. Eur. J. Org. Chem. 2001, 2947-2954).

In the patent publication EP0300861, it has been described that the formylation of aromatics is achievable using alkyl formates in the presence of the superacidic system HF_{(I)}/BF_{3(g)}. In a particular example, vanillin was obtained in 45% yield from guaiacol using methyl formate as the formyl source. The main drawback of such a method is the use of very hazardous and toxic substances in high proportions. Moreover, HF/BF₃ is a gas that imposes to work under important pressure and a high excess, for instance 40 molar eq. HF for 1 molar eq. of guaiacol. Furthermore, this reaction leads to the production of high content of by-products, as demonstrated in Saint-Jalmes, L.; Rochin, C.; Janin, R.; Morel, M. Industrial Chem. Library 1996, 8, 325-335.

An alternative strategy to the direct formylation of hydroxyarenes is the Fries rearrangement of aryl formates. Very little is known on the subject and it is only recently that the reaction as well as its mechanism have been investigated with Lewis acids (Ziegler, G.; Haug, E.; Frey, W.; Kantlehner, W. Z. Naturforsch. 2001, 56b, 1178-1187. Bagno, A.; Kantlehner, W.; Kress, R.; Saielli, G.; Stoyanov, I. J. Org. Chem. 2006, 71, 9331-9340. Bagno, A.; Kantlehner, W.; Saielli, G. J. Phys. Org. Chem. 2008, 21, 682-687). For instance, Ziegler and coworkers obtained a 1:1 mixture of ortho and para-isomers, with a combined yield 20%, when treating 3-methoxyphenyl formate with 4 equiv of triflic acid in 1,2-dichloroethane; main product of the reaction being the deformylated product 3-methoxyphenol.

### Invention

It appears that it is perfectly possible to produce vanillin or vanillin derivatives from guaiacol or a guaicaol derivative carrying an aldehyde function by operating a regio- and chemo-selective formylation, without wishing to be bound by any existing theory. Such a reaction is made according to the present invention with the use of a superacid. The process of the present invention permits to obtain the compound of formula (III) as defined below carrying an aldehyde function in para position relative to the hydroxyl function of phenol compound with a sufficient yield. Such a compound of formula (III) is obtained according to the reaction without the significant presence of isomers such as ortho or meta derivatives of compound (III). Superacids of the present invention also permit to carry out the process in mild conditions.

Thereof, the present invention concerns a process for the production of a compound of formula (III) in which at least a compound of formula (I) and optionally a compound of formula (II) is (are) reacted with a superacid:

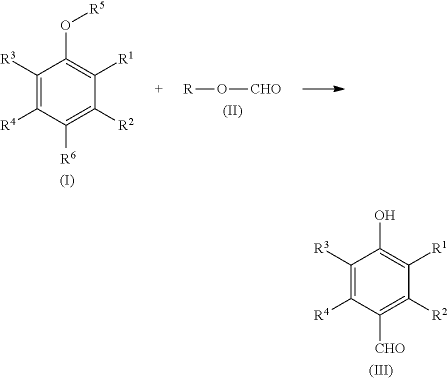

Wherein:
R¹ represents a hydrogen atom or an alkyl, alkenyl or alkoxy group;
R², R³ or R⁴ independently of one another preferably represent a hydrogen atom or an alkyl group;
R⁵ represents a hydrogen atom, an alkyl group or a —CHO group;
R⁶ represents a hydrogen atom or a labile group able to leave compound of formula (I) during the reaction;
R represents a hydrogen atom, an alkyl group or an aryl group; and with the proviso that when compound (I) is used in the reaction without the compound (II), R⁵ is a —CHO group.

The present invention also concerns a compound (III) susceptible to be obtained according to the process of the invention.

It has to be noticed that it's perfectly possible to use several compounds of formula (I) and optionally several compounds of formula (II) during the reaction of the present invention.

“Alkyl” as used herein means a straight chain or branched saturated aliphatic hydrocarbon. Preferably alkyl group comprises 1-18 carbon atoms. Representative saturated straight chain alkyls include methyl, ethyl, n-propyl, n-butyl, n-pentyl, and the like; while saturated branched alkyls include iso-propyl, sec-butyl, iso-butyl, tert-butyl, iso-pentyl, and the like.

“Aryl” as used herein means a 6-carbon monocyclic or 10-carbon bicyclic aromatic ring system wherein 0, 1, 2, 3, or 4 atoms of each ring are substituted. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

“Alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Representative unsaturated straight chain alkenyls include ethenyl, propenyl, butenyl, pentenyl, hexenyl, heptenyl, octenyl, nonenyl, decenyl and the like.

“Alkoxy” as used herein is O-alkyl, wherein alkyl is as defined above. Alkoxy may be for example methoxy, ethoxy, n-propoxy, iso-propoxy, n-butoxy, sec-butoxy, iso-butoxy, and tert-butoxy.

According to the classical definition, a superacid is an acid with an acidity greater than that of 100% pure sulfuric acid, which has a Hammett acidity function (H₀) of −12 (Gillespie, R. J.; Peel, T. E. J. Am. Chem. Soc. 1973, 95, 5173-5178).

Superacids may be used during the reaction as homogeneous or heterogeneous catalysts. Superacids of the invention are preferably in a liquid form or in a solid form, in the conditions of the reaction.

Superacids of the present invention may be those ones having a piKₐ inferior or equal to −2 in dicholorethane, preferably a piKₐ inferior or equal to −10.5 in dicholorethane, according to the method of J. Org. Chem 2011, 76, 391-395 “Equilibrium Acidities of Superacids” Agnes Kutt et al, using an UV-vis spectrophotometric titration.

Preferred superacids of the present invention are notably Brønsted acids, more preferably those ones of fluoro sulfonic group or (per)fluoroalkanesulfonic group.

Superacids of the present invention are preferably compounds carrying at least a fluoro sulfonic group or a (per)fluoroalkanesulfonic group.

Superacids may be chosen in the group comprising: trifluoromethanesulfonic acid (CF₃SO₃H), also known as triflic acid, and fluorosulfonic acid (FSO₃H).

Superacids of the invention may also be compounds carrying at least a sulfate group.

Superacids may be supported on a carrier, such as for example one of the oxides, carbons or organic or inorganic resins. Notably, the carrier may be selected from the group consisting of silica, alumina, zirconia, titania, ceria, magnesia, lanthania, niobia, yttria, zeolite, perovskite, silica clay, and iron oxide and mixtures thereof.

The Superacids may be supported on a carrier in any convenient fashion, particularly by adsorption, ion-exchange, grafting, trapping, impregnation, or sublimation.

R¹ preferably represents methoxy or ethoxy. R² preferably represents hydrogen. R³ preferably represents hydrogen. R⁴ preferably represents hydrogen. R⁵ preferably represents CHO or hydrogen.

As previously defined, R⁶ represents H or a labile group able to leave compound of formula (I) during the reaction, it notably means in the acidic medium of the reaction with action of the superacid. In one aspect, R⁶ may be —SiR¹₃, carboxylic acid or ester or a boron containing group such as —BR⁷₂. R¹ is defined above and R⁷ may represent a hydroxyl, alkyl or alkoxy group. A preferred labile group is a trialkyl silyl group. Another may be a boronic acid which can undergo protodeboration, notably in a superacidic medium.

Compound of formula (I) is preferably chosen in the group comprising: guaiacol, guaiacol formate, phenyl formate, phenol, veratrol, catechol, para-trimethylsilyl guaiacol, guetol (2 ethoxyphenol) and guetol formate.

R preferably represents hydrogen, aryl such as phenyl, guaiacyl, guetyl or alkyl such as methyl.

Compound of formula (II) is preferably chosen in the group comprising: guaiacol formate, formic acid, 2,4,6-trimethylphenol formate, phenyl formate, guetol formate and methyl formate.

Compound of formula (III) is preferably chosen in the group comprising: vanillin and para-hydroxy benzaldehyde, ethylvanillin, veratraldehyde, and 3,4-dihydroxybenzaldehyde.

Reaction of the present invention permits to produce para isomer compound of formula (III) with relatively low amount of meta and/ortho isomers thereof. Preferably, the molar ratio of para/(meta+ortho) is comprised between 5 and 100; para is the para isomer of compound (III), meta is the meta isomer of compound (III) when —CHO group is in position 2 relative to the hydroxyl function, ortho is the ortho isomer of compound (III) when —CHO group is in position 1 relative to the hydroxyl function.

According to the process of the invention, yield of compound (III) may be comprised between 5 and 80 molar %.

Without any limitation, the following reactions may be carried out according to the process of the present invention:

Guaiacol formate (I)→vanillin (III)
Guaiacol (I)+guaiacol formate (II)→vanillin (III)
Guaiacol (I)+formic acid (II)→vanillin (III)
Guaiacol (I)+methyl formate (II)→vanillin (III)
Guaiacol (I)+mesityl formate (II)→vanillin (III)
Phenyl formate (I)→para-hydroxy benzaldehyde (III)
Phenol (I)+phenyl formate (II)→para-hydroxy benzaldehyde (III)
Phenol (I)+formic acid (II)→para-hydroxy benzaldehyde (III)
Guetol (I)+guetol formate (II)→ethyl vanillin (III)
Guetol (I)+formic acid (II)→ethyl vanillin (III)
Guetol (I)+methyl formate (II)→ethyl vanillin (III)
Guetol (I)+mesityl formate (II)→ethyl vanillin (III).

Beside there is no need of a specific solvent during the reaction, it is perfectly possible to use a solvent. Compound (II) as example may be used as solvent.

It has to be noticed that as known by a person skilled in the art, solvents used in the reaction must not amend parameters of the process, such as for example regio-selectivity to obtain compound of formula (III), molar ratio of isomers and/or yield.

Other interesting solvents could be non-coordinating solvents, aprotic solvents, or low polar solvents, such as toluene, benzene, or chlorinated solvents, for example 1,2-dichloroethane, dichloromethane, chloroform, and CCl₄.

Useful solvents are preferably those ones able to dissolve at least compound of formula (I).

Preferably, the medium of the reaction used in the present process of the invention is substantially free or, in some cases, completely free of water, at the start of the reaction. As used herein, the term “substantially free” when used with reference to the absence of water in the medium of the present invention, means that the medium comprises less than 0.1% wt of water, based on the total weight of the medium, notably at the beginning of the reaction; and preferably during the reaction. As used herein, the term “completely free” when used with reference to the absence of water in the medium of the present invention, means that the medium comprises no water at all.

Temperature of the reaction process is preferably comprised between −60 and +80° C., more preferably between −20 and +40° C.

There is no need of a specific pressure during the reaction of the invention, though the process may be optionally carried out under pressure, such as between 1 and 3 bar for example.

Molar proportions of the compounds (I), (II) and superacid may be as follows:
Compound (I): 1
Compound (II): 0-20, and
Superacid: 0.1-20, preferably 0.1-15, more preferably 0.5-10.

When compound (I) is solely used to produce compound (III), molar proportions are preferably as follows:
Compound (I): 1; and
Superacid: 1-5.

Molar ratio of superacid/compound (II) is preferably superior or equal to 0.9, more preferably superior or equal to 1, and highly preferably superior or equal to 2.

In a typical embodiment of the method of the present invention, time of the reaction to produce compound (III) is preferably comprised between 1 minute and 2 hours.

This reaction may be conducted in any conventional equipment suitable to effect production of compound (III). This reaction may be carried out in a continuous or a discontinuous fashion. For example, suitable equipments include a stirred tank or loop reactor.

In a batch process, compounds (I), (II) and superacid may be added and mixed together. It is also possible to first add compounds (I) and (II) and then to further proceed to an addition of superacid to start the reaction. Compound (II) may be used as solvent of compound (I) and then in this way it is necessary first to dissolve compound (I) in compound (II) and then to add superacid.

The efficiency of the process of the present invention can be monitored by any conventional analytical means, such as Infrared spectroscopy, NMR, Raman spectroscopy, GC and HPLC.

At the end of the reaction, superacid may be optionally neutralized and/or removed by distillation, extraction or washings. Said superacids may notably be recycled to the reactor. Compound (III) of interest can be purified by well known methods of the technical field, such as distillation or crystallization.

The following examples are included to illustrate embodiments of the invention. It should be appreciated by those skilled in the art that the techniques disclosed in the following examples represent techniques discovered by the inventor to function well in the practice of the invention. However, those skilled in the art should, in light of the present disclosure, appreciate that many changes can be made in the specific embodiments which are disclosed and still obtain a like or similar result without departing from the concept, spirit and scope of the invention. All such similar substitutes and modifications apparent to those skilled in the art are deemed to be within the spirit, scope and concept of the invention as defined by the appended claims.

### Experimental Part

### Methodology

Analyses by HPLC were made with a Kromasil KR100-5C18, L=250 mm, 0=4.6 mm, Particle Size=5 μm, Pore Size=100 Å (reverse phase) column. Mobile Phase: 1% AcOH in water and MeCN.

### EXAMPLE 1

Guaiacol (9.9 g, 80 mmol, 1 equiv) and 2,4,6-trimethylphenol formate (26.4 g, 160 mmol, 2 equiv) were dissolved in 1,2-dichloroethane (1,2-DCE) (200 mL) at room temperature. CF₃SO₃H (28.4 mL, 320 mmol, 4 equiv) was added to the mixture and the resulting colored solution was stirred at room temperature for 2 h. CF₃SO₃H was then quenched with pyridine (25.8 mL, 320 mmol, 4 equiv) with external cooling in an ice bath and then water (200 mL) was added. The phases were separated, and the aqueous further extracted with dichloromethane (3 times with 50 mL). The combined organic extracts were then washed with brine, dried over Na₂SO₄, filtered and the solvent removed under reduced pressure to give a yellowish oil (40 g). This was submitted to flash chromatography on silica gel eluting with petroleum ether/ethyl acetate 10:1 (v/v), followed by 5:1 (v/v) and finally 2:1 (v/v). Different fractions were collected and fraction 4 (1.3 g) contained mainly the desired vanillin (50% molar). Molar ratio of para/(meta+ortho) at the end of the reaction is equal to 13. 160 mg were purified by preparative HPLC to afford pure VA.

### EXAMPLE 2

In a capped vial, guaiacol formate (304 mg, 2 mmol, 1 equiv) was dissolved in toluene (2.5 mL) and was cooled down to 0° C. in an ice bath. Pre-cooled CF₃SO₃H (0.35 mL, 4 mmol, 2 equiv) at 0° C. in an ice bath was added rapidly to the mixture at 0° C. and the resulting light yellow solution turned slowly to orange then light purple over the time. HPLC yields after 150 min: vanillin: 19.1% molar, iso-vanillin: 0.5% molar, ortho-isomer: 0% molar. Molar ratio para/meta (vanillin/iso-vanillin) is then equal to 37.

### EXAMPLE 3

In a capped vial, guaiacol formate (304 mg, 2 mmol, 1 equiv) was cooled down to 0° C. Pre-cooled CF₃SO₃H (0.35 mL, 4 mmol, 2 equiv) at 0° C. in an ice bath was added rapidly at 0° C. and the resulting very viscous solution was stirred vigorously. HPLC yields after 40 min: vanillin: 6.5% molar, iso-vanillin: 0.2% molar, ortho-isomer: 0% molar. Molar ratio para/meta (vanillin/iso-vanillin) is then equal to 33.

### EXAMPLE 4

In a capped vial, guaiacol (303 mg, 2.4 mmol, 1 equiv) and formic acid (185 mg, 4 mmol, 2 equiv) were dissolved in dichloromethane (5 mL). CF₃SO₃H (0.70 mL, 8 mmol, 4 equiv) was added rapidly to the mixture at room temperature. HPLC yields after 225 min: vanillin: 5.2% molar, iso-vanillin: 0.18% molar, ortho-isomer: 0% molar. Molar ratio para/meta (vanillin/iso-vanillin) is then equal to 29.

### EXAMPLE 5

In a capped vial, guaiacol (132.2 mg, 1.1 mmol, 1 equiv) was dissolved in methyl formate (324.4 mg, 5.4 mmol, 5 equiv). CF₃SO₃H (1.9 mL, 21.47 mmol, 20.2 equiv) was added rapidly to the mixture at room temperature. HPLC yields after 20 h: vanillin: 10.4% molar, iso-vanillin: 0.95% molar, ortho-isomer: 0% molar. Molar ratio para/meta (vanillin/iso-vanillin) is then equal to 11.

1. A process for producing a compound of formula (III), comprising carrying out a reaction of at least a compound of formula (I) and optionally a compound of formula (II) with a superacid: 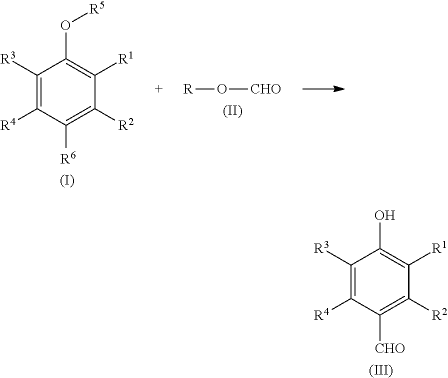
Wherein:
R¹ represents a hydrogen atom, an alkyl group, an alkenyl group, or an alkoxy group;
R², R³ or R⁴ independently of one another represent a hydrogen atom or an alkyl group;
R⁵ represents a hydrogen atom, an alkyl group, or a —CHO group;
R⁶ represents a hydrogen atom or a labile group able to leave said compound of formula (I) during the reaction; and
R represents a hydrogen atom, an alkyl group, or an aryl group; and
with the proviso that when said compound (I) is used in the reaction without the compound (II), R⁵ is a —CHO group.

2. The process according to claim 1, wherein said superacid has a pKₐ inferior or equal to −2 in dicholorethane, said pKₐ being measured according to the method of J. Org. Chem 2011, 76, 391-395 “Equilibrium Acidities of Superacids” Agnes Kutt et al, using an UV-vis spectrophotometric titration.

3. The process according to claim 1, wherein said superacid has a pKₐ inferior or equal to −10.5 in dicholorethane, said pKₐ being measured according to the method of J. Org. Chem 2011, 76, 391-395 “Equilibrium Acidities of Superacids” Agnes Kutt et al, using an UV-vis spectrophotometric titration.

4. The process according to claim 1, wherein said superacid is used as homogeneous or heterogeneous catalyst.

5. The process according to claim 1, wherein said superacid is in a liquid form or in a solid form, in the conditions of the reaction.

6. The process according to claim 1, wherein said superacid is a Brønsted acid.

7. The process according to claim 6, wherein said superacid is a Brønsted acid of a fluoro sulfonic group or a (per)fluoroalkanesulfonic group.

8. The process according to claim 1, wherein said superacid is a compound carrying at least a fluoro sulfonic group or a (per)fluoroalkanesulfonic group.

9. The process according to claim 1, wherein said superacid is selected from the group consisting of trifluoromethanesulfonic acid and fluorosulfonic acid.

10. The process according to claim 1, wherein said superacid is a compound carrying at least a sulfate group.

11. The process according to claim 1, wherein said superacid is supported on a carrier.

12. The process according to claim 1, wherein said compound (I) is selected from the group consisting of: guaiacol, guaiacol formate, phenyl formate, phenol, veratrol, catechol, para-trimethylsilyl guaiacol, guetol, and guetol formate.

13. The process according to claim 1, wherein said compound (II) is used in the reaction and selected from the group consisting of: guaiacol formate, formic acid, 2,4,6-trimethylphenol formate, phenyl formate, guetol formate, and methyl formate.

14. The process according to claim 1, wherein said compound (III) is selected from the group consisting of: vanillin, and para-hydroxy benzaldehyde, ethylvanillin, veratraldehyde, and 3,4-dihydroxybenzaldehyde.

15. The process according to claim 1, wherein the compound (III) comprises a molar ratio of para/(meta+ortho) between 5 and 100; para being the para isomer of said compound (III), meta being the meta isomer of said compound (III), and ortho being the ortho isomer of said compound (III).

16. The process according to claim 1, wherein a yield of said compound (III) is comprised between 5 and 80 molar % is obtained.

17. The process according to claim 1, wherein said reaction is selected from the group consisting of the following reactions:
Guaiacol formate (I)→vanillin (III);
Guaiacol (I)+guaiacol formate (II)→vanillin (III);
Guaiacol (I)+formic acid (II)→vanillin (III);
Guaiacol (I)+methyl formate (II)→vanillin (III);
Guaiacol (I)+mesityl formate (II)→vanillin (III);
Phenyl formate (I)→para-hydroxy benzaldehyde (III);
Phenol (I)+phenyl formate (II)→para-hydroxy benzaldehyde (III);
Phenol (I)+formic acid (II)→para-hydroxy benzaldehyde (III);
Guetol (I)+guetol formate (II)→ethyl vanillin (III);
Guetol (I)+formic acid (II)→ethyl vanillin (III);
Guetol (I)+methyl formate (II)→ethyl vanillin (III); and
Guetol (I)+mesityl formate (II)→ethyl vanillin (III).

18. The process according to claim 1, wherein the reaction is carried out in a medium substantially free of water, at the start of the reaction.

19. The process according to claim 1, wherein molar proportions of the compounds (I), (II) and superacid are as follows:
Compound (I): 1
Compound (II): 0-20, and
Superacid: 0.1-20.

20. The process according to claim 1, wherein, when said compound (I) is solely used to produce said compound (III), molar proportions of said compound (I) and said superacid are as follows:
Compound (I): 1; and
Superacid: 1-5.

21. The process according to claim 1, wherein, when said compound (II) is used, a molar ratio of superacid/compound (II) superior or equal to 0.9 is used.

22. (canceled)
